(19) United States
(12) Patent Application Publication
(10) Pub. No.: US 2026/0097211 A1
(43) Pub. Date: Apr. 9, 2026

(54) HEALTH CONDITION TREATMENT VIA ADAPTIVE LOOP STIMULATION PATCH

(71) Applicant: The Regents of the University of California, Oakland, CA (US)

(72) Inventors: Michelle Khine, Irvine, CA (US); Kurt Qing, Orange, CA (US)

(21) Appl. No.: 19/353,883

(22) Filed: Oct. 9, 2025

Related U.S. Application Data
(60) Provisional application No. 63/705,192, filed on Oct. 9, 2024.

(51) Int. Cl. A61N 1/36; A61B 5/0205; A61B 5/256; A61N 1/04; A61N 1/372

(52) U.S. Cl. CPC A61N 1/36139; A61B 5/0205; A61B 5/256; A61N 1/0484; A61N 1/36103; A61N 1/37282

(57) ABSTRACT

A system for real-time management of a health condition in a patient. The system includes a wearable sensor including at least one sensor for measuring physiological responses, electrodes configured to stimulate and record a nerve, and a sensor processing module for receiving the physiological responses and the nerve response and transmitting them to a computing device, as well as actuate the electrodes to stimulate the nerve. The system further includes the computing device configured to actuate the sensor device to deliver an initial stimulation, receive the physiological responses and the nerve response, generate a response profile specific to the patient, actuate the sensor device to deliver subsequent stimulations according to the response profile, receive subsequent physiological responses and a subsequent nerve response, and adjust, based on a change, the response profile. The steps of subsequent stimulation, receiving responses, and adjusting the response profile may be repeated until a target point.

### CROSS-REFERENCE TO RELATED APPLICATIONS

This application is a non-provisional and claims benefit of U.S. Provisional Application No. 63/705,192 filed Oct. 9, 2024, the specification of which is incorporated herein in its entirety by reference.

### FIELD OF THE INVENTION

The present invention is directed to a medical device for nonpharmacological reduction in blood pressure using closed-loop neurostimulation and personalized parameters.

### BACKGROUND OF THE INVENTION

Hypertension—defined as systolic blood pressure (SBP) >140/90 mmHg or diastolic blood pressure (DBP) >80 mmHg—was a primary or contributing cause of nearly 700,000 deaths in the U.S. in 2021 alone. Almost one-half of all adults in the U.S. (116 million people) and >1 billion people worldwide suffer from hypertension. Hypertension-associated complications encompass one of the most important causes of premature death worldwide. Importantly, this also makes hypertension the most modifiable universal risk factor for cardiovascular disease.

Nearly 20% of U.S. adults who are prescribed anti-hypertensive medication (10.3 million people) suffer from apparent treatment-resistant hypertension (aTRH). Resistant hypertension (RHT) is blood pressure (BP) that remains above the recommended level, despite treatment with ≥3 antihypertensive drugs of different classes. Compared with patients whose BP is controlled, the cardiovascular mortality rate is more than 2× higher in these patients. Just a 10-mm Hg reduction in SBP is associated with a 15%-20% reduction in the risk of coronary artery disease and a 25% to 30% reduction in the risk of stroke. The prevalence of aTRH was 17.7% and 19.7% according to the 2008 and 2018 Scientific Statement definitions, respectively (Δ=2.0%; 95% CI 1.5%, 2.7%). Overall, 10.3 million US adults had aTRH according to the 2018 Scientific Statement. The prevalence of aTRH among patients with predialysis chronic kidney disease is 2-3 times higher than in the general population

Over the past decade, hypertension control in the U.S. has deteriorated, with persistent and even growing disparity of increased hypertension prevalence and worse BP control among minority racial and ethnic subgroups. Even after adjusting for socioeconomic status and health care access, Black/African Americans are 12% less likely to have adequately controlled BP. Long-established race-based pharmacologic treatment guidelines for hypertension are now being debunked as ineffective. Instead, a more precise, personalized, data-driven approach to hypertension control is critically needed. Failure to address hypertension has been associated with higher rates of cardiovascular complications (stroke, heart failure, chronic kidney disease, etc.). Furthermore, 5% of all hypertension and 25% of aTRH have a secondary cause.

Researchers have combined electronic health record system data from 3 large health systems serving 2,420,468 adults with 2 or more outpatient visits that included both blood pressure (BP, mm Hg) values and medication reconciliation. Of the 1,343,489 adults with a diagnosis of hypertension, 113,992 (8.5%) met criteria for aTRH at a BP threshold of 130/80 and 8.3% at a BP threshold of 140/90. This prevalence of aTRH was substantially lower than the 12% to 15% reported previously (2003-2008 and 1988-2008).

Anti-hypertensive drugs includes ACE inhibitors (angiotensin-converting enzyme inhibitors), ARBs (angiotensin II receptor blockers), Beta blockers, Calcium channel blockers, Diuretics Combination drugs, direct renin inhibitors, alpha blockers, PDE-5 inhibitors, stimulators of soluble guanylate cyclase, endothelin receptor antagonists, vasodilators, prostacyclin receptors, and others. However, the most common of these medications are replete with side effects. Angiotensin-converting enzyme inhibitors can cause lightheadedness or dizziness if blood pressure becomes too low, a cough in about 15% of patients, and worsened kidney function in some patients. Beta blockers may cause a slow heart rate, also known as bradycardia, which can cause dizziness, lightheadedness, or tiredness. Additionally, beta blockers may cause cold hands and feet by affecting blood supply to the extremities, as well as insomnia, nightmares, or other sleep changes, nausea and vomiting, sexual dysfunction, blurred vision, and dry mouth or eyes. Thiazide diuretic may cause electrolyte disturbances, hyperuricemia, cholestasis, dermatitis, and/or vasculitis. ARBs generally have a better side effect profile but can still cause dizziness and renal impairment. Calcium channel blockers may lead to peripheral edema and constipation.

The economic burden of hypertension is reported to be more than $120.0 billion in the U.S. alone, while the global cost burden is more than $400.0 billion. This cost burden is due to expenditures associated with cardiovascular complications or conditions for which hypertensives are considered at higher risk and other comorbidities that may raise the cost of medical care. Drug adherence is maintained through SMS reminders, phone apps, support teams (nurse, health managers, pharmacists and other stakeholders), and the Internet. However, recent reports of hacking into insulin pumps worn by diabetics show that it is imperative to regulate all kinds of hardware-based devices and even simple software applications to ensure patient safety and enhance output and data security.

While lack of adherence to prescribed antihypertensive medications is common in patients with aTRH, both apparent and true RHT patients (those who have uncontrolled BP despite confirmed medication adherence) frequently exhibit increased sympathetic activity. As such, over the past 15 years, much progress has been made in nonpharmacological neuromodulation to chronically suppress sympathetic activity, as pharmacological therapies targeting increased sympathetic activity have not been well tolerated due to the undesired effects on chronotropic response resulting in lowering of the heart rate (HR). The prevailing approaches have been electrical activation of the carotid baroreflex and catheter-based renal nerve ablation.

While both approaches have demonstrated suppression in sympathetic activity and BP lowering, without a negative chronotropic response, these highly invasive approaches necessitate surgical implantation and have significant side effects. In recent clinical trials, critical endpoints for safety and efficacy have not yet been achieved. There remains an unmet clinical need for an effective, non-invasive approach to lowering BP in RHT patients; there is an even greater public health need to develop such an approach to prevent the 59 million people in the U.S. who currently have prehypertension from becoming hypertensive.

Studies have demonstrated the effectiveness of stimulating the median nerve for reducing cardiac metabolic demand and lowering BP, with no effect on HR, in rats as well as in humans. However, studies have only demonstrated either transient reductions in BP or open-loop chronic hypertension electro-stimulation based on empirically pre-selected input parameters. Because BP changes from beat to beat, open-loop systems are unable to respond to the inherently dynamic nature of BP, thereby lacking the ability to provide timely, optimized protocols. For example, excessive stimulation can compromise the therapeutic effect and trigger complications. In addition, the major commercially available neurostimulation devices do not record the neural response and cannot account for variable responses to the stimulus among individuals or adjust for variable states such as resting, active, and sleep. In contrast, a closed-loop system would directly and continuously monitor and accordingly adjust stimulus parameters in real-time, achieving more effective and personalized clinical treatment effects while minimizing excessive stimulation avoiding potentially deleterious effects.

Closed-loop systems involving nerve stimulation exist, but fail to provide accurate measurements, and subsequently accurate adjustments to the stimulation for adjusting a physiological characteristic. For example, prior systems exist that stimulate a nerve and measure a nerve response alone. However, these systems fail to provide the calculations necessary to properly adjust any physiological parameter and can only be used for muscle strength measurements. Additionally, prior systems exist that stimulate a nerve and measure a physiological parameter alone. However, these systems fail to provide an efficient method for addressing a health condition through these measurements and adjustments alone. Additionally, prior systems exist with implantable sensors for measuring multiple parameters and adjusting stimulation accordingly. However, the implantable requirement of these sensors reduces the overall usability of the system for continuous monitoring and therapy. Thus, there exists a present need for an accurate and efficient closed-loop system for nerve stimulation, measurement, and response for addressing health conditions in a patient.

### BRIEF SUMMARY OF THE INVENTION

It is an objective of the present invention to provide systems and methods that allow for nonpharmacological reduction in blood pressure using closed-loop neurostimulation and personalized parameters, as specified in the independent claims. Embodiments of the invention are given in the dependent claims. Embodiments of the present invention can be freely combined with each other if they are not mutually exclusive.

The present invention features a system for real-time management of a health condition in a patient. The system may comprise a wearable sensor device comprising at least one sensor for measuring physiological responses of the patient, electrodes configured to stimulate a nerve, and to record a nerve response from the nerve. The device may further comprise a sensor processing module coupled to the sensor and the electrodes, configured to receive the physiological responses and the nerve response from the sensor and the electrodes and transmit them to a computing device, as well as actuate the electrodes to stimulate the nerve. The system may further comprise the computing device coupled to the sensor device. The computing device may be configured to actuate the wearable sensor device to deliver an initial stimulation to the nerve, receive the physiological responses and the nerve response from the sensor device, and generate a response profile specific to the patient. The computing device may be further configured to actuate the wearable sensor device to deliver subsequent stimulations to the nerve according to the response profile, receive subsequent physiological responses and a subsequent nerve response, and adjust, based on a change in the subsequent physiological responses and the subsequent nerve response, the response profile. The steps of subsequent stimulation, receiving responses, and adjusting the response profile may be repeated until the subsequent physiological responses meet a target value. In some embodiments, the processing and closed-loop features may be implemented in the wearable sensor device, the computing device, or a combination thereof.

By innervating the median nerve through closed-loop transcutaneous electrical stimulation, the present invention is configured to control RHT and even prevent the onset of hypertension, subsequently preventing downstream cardiovascular and related diseases. This proposed Health Condition Treatment via Adaptive Loop Stimulation (HEALS) Patch would allow the monitoring, modulation, and optimization of nerve stimulation signals in real-time, unlike any other non-invasive approach. Unlike any system that currently exists, we will record continuous BP and HR, as well as the median nerve evoked potential to ensure effective and optimized dosing.

One of the unique and inventive technical features of the present invention is the implementation of a closed-loop system for nerve stimulation and analysis of physiological parameters and nerve responses. Without wishing to limit the invention to any theory or mechanism, it is believed that the technical feature of the present invention advantageously provides for personalized, precise, real-time treatment of a plurality of different health conditions. None of the presently known prior references or work has the unique inventive technical feature of the present invention.

Furthermore, the inventive technical feature of the present invention is counterintuitive. The reason that it is counterintuitive is because it contributed to a surprising result. One of ordinary skill of the art would expect that the inclusion of nerve response measurement in a closed-loop stimulation system would be unnecessary and inefficient, especially in comparison to prior systems that address this issue with the measurement of non-nerve physiological parameters alone with no suggestion that results would be improved with the addition of nerve response measurement.

Furthermore, one of ordinary skill in the art would not expect that a non-implanted wearable electrode system would not be capable of achieving nerve response measurements to a suitable degree to accurately adjust stimulation for the treatment of health conditions. Surprisingly, the present invention implements a wearable system configured to measure both a physiological parameter and a nerve response to adjust stimulation and address a health condition of a patient. Thus, the inventive technical feature of the present invention contributed to a surprising result and is counterintuitive.

Any feature or combination of features described herein are included within the scope of the present invention provided that the features included in any such combination are not mutually inconsistent as will be apparent from the context, this specification, and the knowledge of one of ordinary skill in the art. Additional advantages and aspects of the present invention are apparent in the following detailed description and claims.

### BRIEF DESCRIPTION OF THE SEVERAL VIEWS OF THE DRAWING(S)

The features and advantages of the present invention will become apparent from a consideration of the following detailed description presented in connection with the accompanying drawings in which:

FIG. 1A shows a schematic diagram of the system for health condition treatment through a closed stimulation and response loop of the present invention.

FIG. 1B shows a flow chart of the method for health condition treatment through a closed stimulation and response loop of the present invention.

FIG. 2 shows a photographic diagram of the system for health condition treatment through a closed stimulation and response loop of the present invention.

FIG. 3 shows a graph of systolic blood pressure (SBP), diastolic blood pressure (DBP), and mean arterial pressure (MAP) at a baseline, as well as in response to various levels of stimulation. The data clearly shows a reduction in each of these values in response to electrical stimulation.

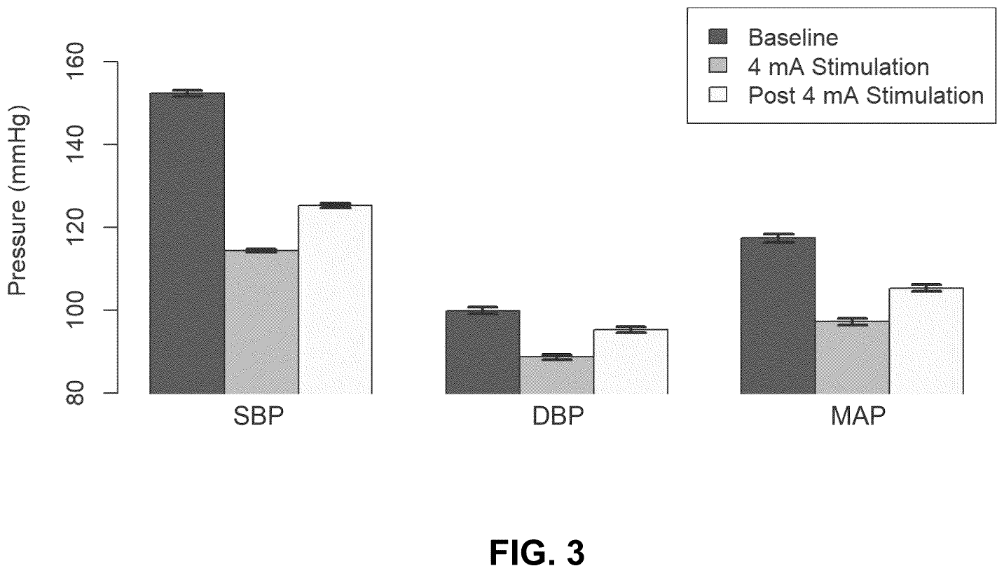

FIG. 4 shows a set of graphs of beat-to-beat blood pressure recording as measured by the system of the present invention.

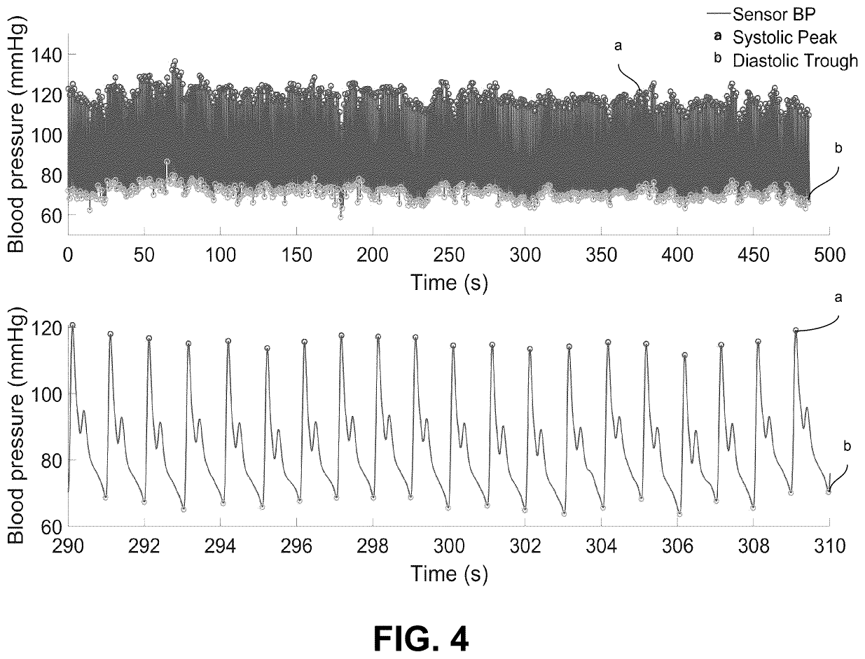

### DETAILED DESCRIPTION OF THE INVENTION

Following is a list of elements corresponding to a particular element referred to herein:
100 system
110 wearable sensor device
111 sensor
112 electrodes
113 stimulation electrode
114 recording electrode
115 sensor processing module
120 computing device
122 processor
124 memory component

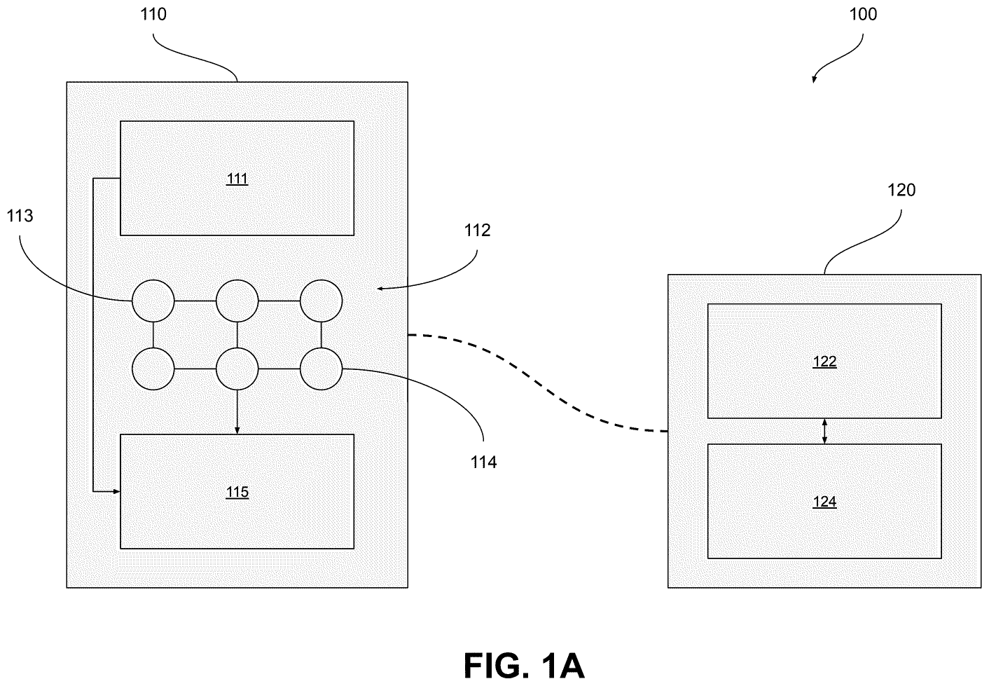

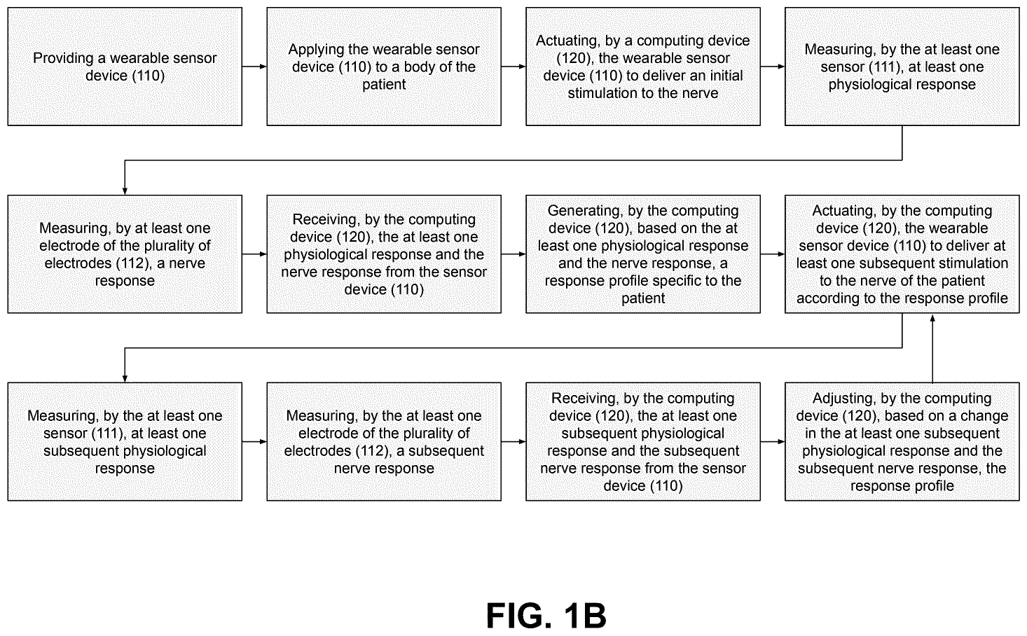

Referring now to FIG. 1A, the present invention features a system (100) for real-time management of a health condition in a patient. The system (100) may comprise a wearable sensor device (110) coupled to a body of the patient. The device (110) may comprise at least one sensor (111) configured to measure at least one physiological response of the patient. The device (110) may further comprise a plurality of electrodes (112) comprising at least one stimulation electrode (113) configured to stimulate a nerve of the patient, and at least one recording electrode (114) configured to record a nerve response from the nerve of the patient, generated in response to stimulation of the nerve. The device (110) may further comprise a sensor processing module (115) communicatively coupled to the at least one sensor (111) and the plurality of electrodes (112), configured to receive the at least one physiological response and the nerve response from the at least one sensor (111) and the at least one recording electrode (114) and transmit the at least one physiological response and the nerve response to a computing device (120), and actuate the at least one stimulation electrode (113) to stimulate the nerve.

The system (100) may further comprise the computing device (120) communicatively coupled to the sensor device (110). The computing device (120) may comprise a processor (122) configured to execute computer-readable instructions, and a memory component (124) operatively coupled to the processor, comprising computer-readable instructions. The computer-readable instructions may comprise actuating the at least one stimulation electrode (113) of the wearable sensor device (110) to deliver an initial stimulation to the nerve of the patient. The computer-readable instructions may further comprise receiving the at least one physiological response and the nerve response from the at least one sensor device (111) and the at least one recording electrode (114) of the wearable sensor device (110). The computer-readable instructions may further comprise generating, based on the at least one physiological response and the nerve response, a response profile specific to the patient.

The computer-readable instructions may further comprise actuating the wearable sensor device (110) to deliver at least one subsequent stimulation by the at least one stimulation electrode (113) to the nerve of the patient according to the response profile. The computer-readable instructions may further comprise receiving at least one subsequent physiological response and a subsequent nerve response from the at least one sensor device (111) and the at least one recording electrode (114) of the wearable sensor device (110). The computer-readable instructions may further comprise adjusting, based on a change in the at least one subsequent physiological response and the subsequent nerve response, the response profile. The computer-readable instructions may further comprise repeating stimulation, measurement, and adjustment until the at least one subsequent physiological response meets a target value indicative of management of the health condition.

In some embodiments, the at least one physiological response may comprise blood pressure, heart rate, heart rate variability, cardiac output, sustained hypertensive episodes, electrical brain activity, skeletal muscle function, electrical heart activity, respiration, oxygenation, chemical levels in blood, chemical levels in sweat, patient input, cerebral perfusion pressure optimization, or a combination thereof. In some embodiments, the computing device (120) may be coupled to the sensor device (110) by a wired connection, a close-range wireless connection, a long-range wireless connection, or a combination thereof. In some embodiments, the computing device (120) may comprise a personal computing device, a portable computing device, or a cloud computing device. In some embodiments, the memory component (124) may further comprise a predictive artificial intelligence (AI) model configured to accept physiological responses and nerve responses as input and generate and adjust the response profile as output. In some embodiments, the nerve may comprise a median nerve, a vagal nerve, or a sciatic nerve. In some embodiments, the health condition may comprise recurrent pain, headache, hypertension, hypertensive events, heart attacks, strokes, anxiety, Post-Traumatic Stress Disorder (PTSD), treating clots/improve circulation, inflammation, motility, or a combination thereof.

Referring again to FIG. 1A, the present invention features a system (100) for real-time management of a health condition in a patient. In some embodiments, the system (100) may comprise a sensor device (110) coupled to a body of the patient. The device (110) may comprise at least one sensor (111) configured to measure a blood pressure measurement of the patient and a heart rate measurement of the patient. The device (110) may further comprise a plurality of electrodes (112) comprising at least one stimulation electrode (113) configured to stimulate a nerve of the patient, and at least one recording electrode (114) configured to record a nerve response from the nerve of the patient, generated in response to stimulation of the nerve. The device (110) may further comprise a sensor processing module (115) communicatively coupled to the plurality of electrodes (112), configured to receive the blood pressure measurement, the heart rate measurement, and the nerve response from the at least one sensor (111) and the at least one recording electrode (114) and transmit the at least one physiological response and the nerve response to a computing device (120), and actuate the at least one stimulation electrode (113) to stimulate the median nerve.

The system (100) may further comprise the computing device (120) communicatively coupled to the sensor device (110). The computing device (120) may comprise a processor (122) configured to execute computer-readable instructions, and a memory component (124) operatively coupled to the processor, comprising computer-readable instructions. The computer-readable instructions may comprise actuating the at least one stimulation electrode (113) of the wearable sensor device (110) to deliver an initial stimulation to the median nerve of the patient. The computer-readable instructions may further comprise receiving the blood pressure measurement, the heart rate measurement, and the nerve response from the at least one sensor device (111) and the at least one recording electrode (114) of the wearable sensor device (110). The computer-readable instructions may further comprise generating, based on the blood pressure measurement, the heart rate measurement, and the nerve response, a response profile specific to the patient.

The computer-readable instructions may further comprise actuating the sensor device (110) to deliver at least one subsequent stimulation by the at least one stimulation electrode (113) to the median nerve of the patient. The computer-readable instructions may further comprise receiving a subsequent blood pressure measurement, a subsequent heart rate measurement, and a subsequent nerve response from the at least one sensor device (111) and the at least one recording electrode (114) of the wearable sensor device (110). The computer-readable instructions may further comprise adjusting, based on a change in the subsequent blood pressure measurement, the subsequent heart rate measurement, and the subsequent nerve response, the response profile. The computer-readable instructions may further comprise repeating stimulation, measurement, and adjustment until the subsequent blood pressure measurement, the subsequent heart rate measurement, or a combination thereof meet a target value indicative of management of the health condition.

In some embodiments, the computing device (120) may be coupled to the sensor device (110) by a wired connection, a close-range wireless connection, a long-range wireless connection, or a combination thereof. In some embodiments, the computing device (120) may comprise a personal computing device, a portable computing device, or a cloud computing device. In some embodiments, the memory component (124) may further comprise a predictive artificial intelligence (AI) model configured to accept blood pressure measurements, heart rate measurements, and nerve responses as input and generate and adjust the response profile as output. In some embodiments, the health condition may comprise recurrent pain, headache, hypertension, hypertensive events, heart attacks, strokes, anxiety, PTSD, treating clots/improve circulation, inflammation, motility, or a combination thereof.

Referring now to FIG. 1B, the present invention features a method for real-time management of a health condition in a patient, the method comprising providing a wearable sensor device (110). The device (110) may comprise at least one sensor (111), a plurality of electrodes (112), and a sensor processing module (115) communicatively coupled to the at least one sensor (111) and the plurality of electrodes (112). The method may further comprise applying the wearable sensor device (110) to a body of the patient such that the plurality of electrodes (112) are able to apply stimulation to and record responses from a nerve. The method may further comprise actuating, by a computing device (120), the wearable sensor device (110) to deliver an initial stimulation to the nerve. The method may further comprise measuring, by the at least one sensor (111), at least one physiological response. The method may further comprise measuring, by at least one electrode of the plurality of electrodes (112), a nerve response. The method may further comprise receiving, by the computing device (120), the at least one physiological response and the nerve response from the wearable sensor device (110). The method may further comprise generating, by the computing device (120), based on the at least one physiological response and the nerve response, a response profile specific to the patient.

The method may further comprise actuating, by the computing device (120), the wearable sensor device (110) to deliver at least one subsequent stimulation to the nerve of the patient according to the response profile. The method may further comprise measuring, by the at least one sensor (111), at least one subsequent physiological response. The method may further comprise measuring, by the at least one electrode of the plurality of electrodes (112), a subsequent nerve response. The method may further comprise receiving, by the computing device (120), the at least one subsequent physiological response and the subsequent nerve response from the wearable sensor device (110). The method may further comprise adjusting, by the computing device (120), based on a change in the at least one subsequent physiological response and the subsequent nerve response, the response profile. The method may further comprise repeating stimulation, measurements, and adjustment until the at least one subsequent physiological response meets a target value indicative of management of the health condition.

In some embodiments, the at least one physiological response may comprise blood pressure, heart rate, heart rate variability, cardiac output, sustained hypertensive episodes, electrical brain activity, skeletal muscle function, electrical heart activity, respiration, oxygenation, chemical levels in blood, chemical levels in sweat, patient input, cerebral perfusion pressure optimization, or a combination thereof. In some embodiments, the computing device (120) may be coupled to the wearable sensor device (110) by a wired connection, a close-range wireless connection, a long-range wireless connection, or a combination thereof. In some embodiments, the computing device (120) may comprise a personal computing device, a portable computing device, or a cloud computing device. In some embodiments, the method may further comprise providing a predictive artificial intelligence (AI) model configured to accept physiological responses and nerve responses as input and generate and adjust the response profile as output, inputting the at least one physiological response and the nerve response into the predictive AI model, generating, by the predictive AI model, the response profile, inputting the at least one subsequent physiological response and the subsequent nerve response into the predictive AI model, and adjusting, by the predictive AI model, the response profile. In some embodiments, the nerve may comprise a median nerve, a vagal nerve, or a sciatic nerve. In some embodiments, the health condition may comprise recurrent pain, headache, hypertension, hypertensive events, heart attacks, strokes, anxiety, PTSD, treating clots/improve circulation, inflammation, motility, or a combination thereof.

The present invention features a continuous BP sensor with additional electrodes for stimulation and recording from the median nerve and a microcontroller. The whole unit wirelessly transmits the nerve response and the real-time BP and HR to a tablet or phone via Bluetooth®. The data is processed and analyzed to establish a response profile for each patient, and the stimulus parameters are manually or automatically optimized to achieve the target BP. The median nerve recording is used to assess the dose-response to the stimulation, and the HR is used to assess autonomic tone and avoid cardiac side effects.

In some embodiments, the present invention may be used for the treatment of any type of recurrent pain, such as that resulting from sickle cell anemia, headache (i.e., dizziness, issues with equilibrium, confusion), hypertension, hypertensive events (e.g., pre-eclampsia), heart attacks, strokes, anxiety, PTSD, treating clots, improving circulation, inflammation, arthritis, motility (i.e., treating acid reflux), or a combination thereof. In some embodiments, the physiological response measured by the at least one sensor (111) may comprise patient input, such as a verbal response, pushing a button, typing a response, etc. In some embodiments, the nerve stimulated and measured by the present invention may comprise any peripheral nerve.

The system of the present invention may be used for treating hypertension in a patient. In this embodiment of the system, the one or more sensors may comprise a continuous blood pressure sensor (e.g., wrist-worn), a heart rate sensor, an electrocardiogram (ECG) sensor, a nerve recording device, or a combination thereof. The sensors may be configured to measure bear-to-beat blood pressure (systolic, diastolic, mean arterial pressure), heart rate, heart rate variability, cardiac rhythm, or a combination thereof. The system may be configured to stimulate the median nerve (e.g., by transcutaneous stimulation at the wrist), but other peripheral nerves may be used. The system may measure compound nerve action potentials (CNAPs), nerve conduction velocity, stimulus-evoked nerve responses, or a combination thereof from the nerve. The system may be configured to stimulate the nerve until the blood pressure is in the range of <140 mmHg for systolic and <90 mmHg for diastolic, optimal mean arterial pressure, reduced blood pressure variability, or a combination thereof. To maintain the target nerve response profile and physiologic parameters (such as BP and HR), the stimulator will automatically adjust the stimulus waveform via on-board algorithms. The nerve response profiles and corresponding physiologic effects are determined via a calibration step that will be performed on initial use and updated as needed over time. Example stimulus waveform changes include pulse width and current amplitude. Calibration should be done by medical providers, and profiles and targets set based on individual needs and safety limits.

The system of the present invention may be used for treating hypertensive events, such as pre-eclampsia. In this embodiment of the system, the one or more sensors may comprise a continuous blood pressure sensor, a heart rate sensor, an ECG sensor, optical flow sensors, or a combination thereof. The sensors may be configured to measure rapid blood pressure changes, sustained hypertensive episodes, heart rate patterns, cardiac output variations, or a combination thereof. The system may be configured to stimulate the median nerve (e.g., through transcutaneous stimulation at the wrist). The system may measure CNAPs, stimulus response amplitude, nerve activation thresholds, or a combination thereof from the nerve. The system may be configured to stimulate the nerve until severe hypertensive episodes >160/110 mmHg, maintenance of a stable blood pressure (e.g., during pregnancy).

The system of the present invention may be used for treating heart attacks. In this embodiment of the system, the one or more sensors may comprise an ECG sensor, a blood pressure sensor, a heart rate sensor, a respiration sensor, a pulse oximeter, or a combination thereof. The sensors may be configured to measure cardiac rhythm abnormalities, blood pressure changes, heart rate variability, ST-segment changes, or a combination thereof. The system may be configured to stimulate the median nerve for automatic modulation. The system may measure CNAPs, autonomic nerve activity modulation, or a combination thereof from the nerve. The system may be configured to stimulate the nerve until cardiac rhythm is stabilized, blood pressure is optimized for cardiac perfusion, reduced cardiac workload, or a combination thereof.

The system of the present invention may be used for treating strokes. In this embodiment of the system, the one or more sensors may comprise blood pressure sensors, heart rate sensors, respiration sensors, ECG sensors, optical flow sensors, or a combination thereof. The sensors may be configured to measure blood pressure control, cerebral perfusion pressure optimization, cardiac rhythm stability, or a combination thereof. The system may be configured to stimulate the median nerve for blood pressure control. The system may measure CNAPs, nerve conduction parameter, or a combination thereof of the nerve. The system may be configured to stimulate the nerve until optimal cerebral perfusion pressure is achieved, blood pressure reduction is controlled (e.g., during an acute stroke), or a combination thereof. In some embodiments, the target values in general may be dependent on the patient.

In some embodiments, the response profile implemented by the present invention may comprise a patient-specific dataset comprising the patient's blood pressure response curves to different stimulation parameters, nerve activation thresholds and compound action potential amplitudes, temporal patterns of physiological responses (e.g., onset time, duration, recovery, variability), dose-response relationships (i.e., stimulation intensity vs. blood pressure reduction), patient-specific optimal stimulation parameters, historical response data for adaptive learning, or a combination thereof. The response profile may be stored locally on the memory component, stored in a cloud-based server to which the system is communicatively coupled, or a combination thereof. The response profile may be accessed in real-time for closed-loop control decisions. The response profile may be retrieved for parameter optimization and treatment planning. The response profile may be used for longitudinal analysis and algorithm refinement.

### EXAMPLE

The following is a non-limiting example of the present invention. It is to be understood that said example is not intended to limit the present invention in any way. Equivalents or substitutes are within the scope of the present invention.

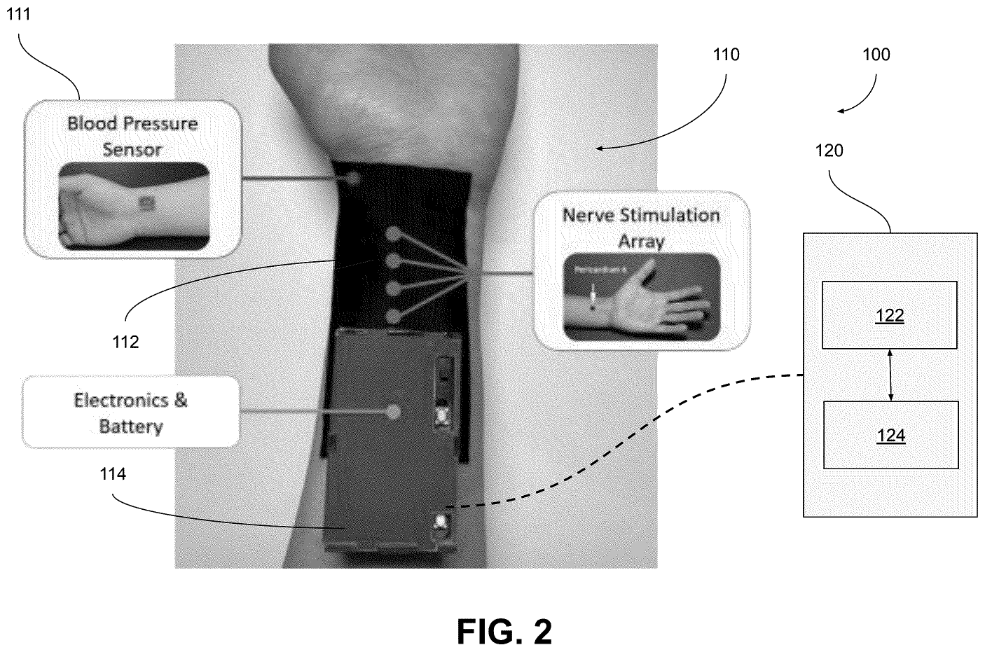

A working example of the health-condition-management system of the present invention has been generated, as seen in FIG. 2. The system was incorporated into an adhesive patch configured to fit onto the arm of a patient. A blood pressure sensor was incorporated into the patch to measure the blood pressure and heart rate of the patient from the patient's vasculature. A nerve stimulation array was also incorporated into the patch to stimulate the median nerve and measure a dose-response. The blood pressure sensor and the nerve stimulation array were both coupled to electronics and a battery held in a housing, configured to transmit the data from the blood pressure sensor and the nerve stimulation array wirelessly to a computing device. The computing device was configured to accept the data from the blood pressure sensor and the nerve stimulation array, process the data, generate a specific response profile for the patient, and continue this process repeatedly, adjusting the response profile over time to bring the physiological responses of the patient to a target level. This working prototype was successful in reducing hypertension in the patient through subsequent stimulations and measurements of nerve responses.

The computer system can include a desktop computer, a workstation computer, a laptop computer, a netbook computer, a tablet, a handheld computer (including a smartphone), a server, a supercomputer, a wearable computer (including a SmartWatch™), or the like and can include digital electronic circuitry, firmware, hardware, memory, a computer storage medium, a computer program, a processor (including a programmed processor), an imaging apparatus, wired/wireless communication components, or the like. The computing system may include a desktop computer with a screen, a tower, and components to connect the two. The tower can store digital images, numerical data, text data, or any other kind of data in binary form, hexadecimal form, octal form, or any other data format in the memory component. The data/images can also be stored in a server communicatively coupled to the computer system. The images can also be divided into a matrix of pixels, known as a bitmap that indicates a color for each pixel along the horizontal axis and the vertical axis. The pixels can include a digital value of one or more bits, defined by the bit depth. Each pixel may comprise three values, each value corresponding to a major color component (red, green, and blue). A size of each pixel in data can range from 8 bits to 24 bits. The network or a direct connection interconnects the imaging apparatus and the computer system.

The term “processor” encompasses all kinds of apparatus, devices, and machines for processing data, including by way of example a programmable microprocessor, a microcontroller comprising a microprocessor and a memory component, an embedded processor, a digital signal processor, a media processor, a computer, a system on a chip, or multiple ones, or combinations, of the foregoing. The apparatus can include special-purpose logic circuitry, e.g., an FPGA (field programmable gate array) or an ASIC (application-specific integrated circuit). Logic circuitry may comprise multiplexers, registers, arithmetic logic units (ALUs), computer memory, look-up tables, flip-flops (FF), wires, input blocks, output blocks, read-only memory, randomly accessible memory, electronically-erasable programmable read-only memory, flash memory, discrete gate or transistor logic, discrete hardware components, or any combination thereof. The apparatus also can include, in addition to hardware, code that creates an execution environment for the computer program in question, e.g., code that constitutes processor firmware, a protocol stack, a database management system, an operating system, a cross-platform runtime environment, a virtual machine, or a combination of one or more of them. The apparatus and execution environment can realize various different computing model infrastructures, such as web services, distributed computing and grid computing infrastructures. The processor may include one or more processors of any type, such as central processing units (CPUs), graphics processing units (GPUs), special-purpose signal or image processors, field-programmable gate arrays (FPGAs), tensor processing units (TPUs), and so forth.

A computer program (also known as a program, software, software application, script, or code) can be written in any form of programming language, including compiled or interpreted languages, declarative or procedural languages, and it can be deployed in any form, including as a stand-alone program or as a module, component, subroutine, object, or other unit suitable for use in a computing environment. A computer program may, but need not, correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, subprograms, or portions of code). A computer program can be deployed to be executed on one computer or on multiple computers that are located at one site or distributed across multiple sites and interconnected by a communication network.

Embodiments of the subject matter and the operations described herein can be implemented in digital electronic circuitry, or in computer software, firmware, or hardware, including the structures disclosed in this specification and their structural equivalents, or in combinations of one or more of them. Embodiments of the subject matter described in this specification can be implemented as one or more computer programs, i.e., one or more modules of computer program instructions, encoded on computer storage medium for execution by, or to control the operation of, a data processing apparatus.

A computer storage medium can be, or can be included in, a computer-readable storage device, a computer-readable storage substrate, a random or serial access memory array or device, or a combination of one or more of them. Moreover, while a computer storage medium is not a propagated signal, a computer storage medium can be a source or destination of computer program instructions encoded in an artificially generated propagated signal. The computer storage medium can also be, or can be included in, one or more separate physical components or media (e.g., multiple CDs, drives, or other storage devices). The operations described in this specification can be implemented as operations performed by a data processing apparatus on data stored on one or more computer-readable storage devices or received from other sources.

Program code embodied on a computer readable medium may be transmitted using any appropriate medium, including but not limited to wireless, wireline, optical fiber cable, R. F, Bluetooth, storage media, computer buses, etc., or any suitable combination of the foregoing. Computer program code for carrying out operations for aspects of the present disclosure may be written in any combination of one or more programming languages, including an object oriented programming language such as Java, Smalltalk, C #, Ruby, or the like, conventional procedural programming languages, such as Pascal, FORTRAN, BASIC, or similar programming languages, programming languages that have both object-oriented and procedural aspects, such as the “C” programming language, C++, Python, or the like, conventional functional programming languages such as Scheme, Common Lisp, Elixir, or the like, conventional scripting programming languages such as PHP, Perl, Javascript, or the like, or conventional logic programming languages such as PROLOG, ASAP, Datalog, or the like.

The program code may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider).

The processes and logic flows described in this specification can be performed by one or more programmable processors executing one or more computer programs to perform actions by operating on input data and generating output. The processes and logic flows can also be performed by, and apparatus can also be implemented as, special purpose logic circuitry, e.g., an FPGA (field programmable gate array) or an ASIC (application-specific integrated circuit).

Processors suitable for the execution of a computer program include, by way of example, both general and special purpose microprocessors, and any one or more processors of any kind of digital computer. Generally, a processor will receive instructions and data from a read-only memory or a random access memory or both. The essential elements of a computer are a processor for performing actions in accordance with instructions and one or more memory devices for storing instructions and data. Generally, a computer will also include, or be operatively coupled to receive data from or transfer data to, or both, one or more mass storage devices for storing data, e.g., magnetic, magneto-optical disks, or optical disks.

However, a computer need not have such devices. Moreover, a computer can be embedded in another device, e.g., a mobile telephone, a personal digital assistant (PDA), a mobile audio or video player, a game console, a Global Positioning System (GPS) receiver, or a portable storage device (e.g., a universal serial bus (USB) flash drive), to name just a few. Devices suitable for storing computer program instructions and data include all forms of non-volatile memory, media and memory devices, including by way of example semiconductor memory devices, e.g., EPROM, EEPROM, and flash memory devices; magnetic disks, e.g., internal hard disks or removable disks; magneto-optical disks; and CD-ROM and DVD-ROM disks. The processor and the memory can be supplemented by, or incorporated in, special purpose logic circuitry.

Computers typically include known components, such as a processor, an operating system, system memory, memory storage devices, input-output controllers, input-output devices, and display devices. It will also be understood by those of ordinary skill in the relevant art that there are many possible configurations and components of a computer and may also include cache memory, a data backup unit, and many other devices. To provide for interaction with a user, embodiments of the subject matter described in this specification can be implemented on a computer having a display device, e.g., an LCD (liquid crystal display), LED (light emitting diode) display, or OLED (organic light emitting diode) display, for displaying information to the user.

Examples of input devices include a keyboard, cursor control devices (e.g., a mouse or a trackball), a microphone, a scanner, and so forth, wherein the user can provide input to the computer. Other kinds of devices can be used to provide for interaction with a user as well; for example, feedback provided to the user can be in any form of sensory feedback, e.g., visual feedback, auditory feedback, or tactile feedback; and input from the user can be received in any form, including acoustic, speech, or tactile input. Examples of output devices include a display device (e.g., a monitor or projector), speakers, a printer, a network card, and so forth. Display devices may include display devices that provide visual information, this information typically may be logically and/or physically organized as an array of pixels. In addition, a computer can interact with a user by sending documents to and receiving documents from a device that is used by the user; for example, by sending web pages to a web browser on a user's client device in response to requests received from the web browser.

An interface controller may also be included that may comprise any of a variety of known or future software programs for providing input and output interfaces. For example, interfaces may include what are generally referred to as “Graphical User Interfaces” (often referred to as GUI's) that provide one or more graphical representations to a user. Interfaces are typically enabled to accept user inputs using means of selection or input known to those of ordinary skill in the related art. In some implementations, the interface may be a touch screen that can be used to display information and receive input from a user. In the same or alternative embodiments, applications on a computer may employ an interface that includes what are referred to as “command line interfaces” (often referred to as CLI's). CLI's typically provide a text based interaction between an application and a user. Typically, command line interfaces present output and receive input as lines of text through display devices. For example, some implementations may include what are referred to as a “shell” such as Unix Shells known to those of ordinary skill in the related art, or Microsoft® Windows Powershell that employs object-oriented type programming architectures such as the Microsoft®.NET framework.

Those of ordinary skill in the related art will appreciate that interfaces may include one or more GUI's, CLI's or a combination thereof. A processor may include a commercially available processor such as a Celeron, Core, or Pentium processor made by Intel Corporation®, a SPARC processor made by Sun Microsystems®, an Athlon, Sempron, Phenom, or Opteron processor made by AMD Corporation®, or it may be one of other processors that are or will become available. Some embodiments of a processor may include what is referred to as multi-core processor and/or be enabled to employ parallel processing technology in a single or multi-core configuration. For example, a multi-core architecture typically comprises two or more processor “execution cores”. In the present example, each execution core may perform as an independent processor that enables parallel execution of multiple threads. In addition, those of ordinary skill in the related field will appreciate that a processor may be configured in what is generally referred to as 32 or 64 bit architectures, or other architectural configurations now known or that may be developed in the future.

A processor typically executes an operating system, which may be, for example, a Windows type operating system from the Microsoft Corporation®; the Mac OS X operating system from Apple Computer Corp. ®; a Unix® or Linux®-type operating system available from many vendors or what is referred to as an open source; another or a future operating system; or some combination thereof. An operating system interfaces with firmware and hardware in a well-known manner, and facilitates the processor in coordinating and executing the functions of various computer programs that may be written in a variety of programming languages. An operating system, typically in cooperation with a processor, coordinates and executes functions of the other components of a computer. An operating system also provides scheduling, input-output control, file and data management, memory management, and communication control and related services, all in accordance with known techniques.

Connecting components may be properly termed as computer-readable media. For example, if code or data is transmitted from a website, server, or other remote source using a coaxial cable, fiber optic cable, twisted pair, digital subscriber line (DSL), or wireless technology such as infrared, radio, or microwave signals, then the coaxial cable, fiber optic cable, twisted pair, DSL, or wireless technology are included in the definition of medium. Combinations of media are also included within the scope of computer-readable media.

The present invention may comprise or implement a neural network for machine learning tasks. The neural network may be stored, trained, and/or executed entirely on the user's computer, partly on the user's computer, as a stand-alone software package, partly on the user's computer and partly on a remote computer or entirely on the remote computer or server. The neural network may be stored in the form of program code, as described above. The neural network, in some embodiments, may be a perceptron neural network, a feed forward neural network, a multilayer perceptron neural network, a convolutional neural network, a radial basis functional neural network, a recurrent neural network, a long short-term memory neural network, a sequence-to-sequence neural network model, a modular neural network, or the like.

Although there has been shown and described the preferred embodiment of the present invention, it will be readily apparent to those skilled in the art that modifications may be made thereto which do not exceed the scope of the appended claims. Therefore, the scope of the invention is only to be limited by the following claims. In some embodiments, the figures presented in this patent application are drawn to scale, including the angles, ratios of dimensions, etc. In some embodiments, the figures are representative only and the claims are not limited by the dimensions of the figures. In some embodiments, descriptions of the inventions described herein using the phrase “comprising” includes embodiments that could be described as “consisting essentially of” or “consisting of”, and as such the written description requirement for claiming one or more embodiments of the present invention using the phrase “consisting essentially of” or “consisting of”is met.

The reference numbers recited in the below claims are solely for ease of examination of this patent application, and are exemplary, and are not intended in any way to limit the scope of the claims to the particular features having the corresponding reference numbers in the drawings.

What is claimed is:

1. A system (100) for real-time management of a health condition in a patient, the system (100) comprising:
a. a wearable sensor device (110) coupled to a body of the patient, the device (110) comprising:
i. at least one sensor (111) configured to measure at least one physiological response of the patient;
ii. a plurality of electrodes (112) comprising:
A. at least one stimulation electrode (113) configured to stimulate a nerve of the patient; and
B. at least one recording electrode (114) configured to record a nerve response from the nerve of the patient, generated in response to stimulation of the nerve; and
iii. a sensor processing module (115) communicatively coupled to the at least one sensor (111) and the plurality of electrodes (112), configured to receive the at least one physiological response and the nerve response from the at least one sensor (111) and the at least one recording electrode (114) and transmit the at least one physiological response and the nerve response to a computing device (120), and actuate the at least one stimulation electrode (113) to stimulate the nerve; and
b. the computing device (120) communicatively coupled to the sensor device (110), comprising:
i. a processor (122) configured to execute computer-readable instructions; and
ii. a memory component (124) operatively coupled to the processor, comprising computer-readable instructions for:
A. actuating the at least one stimulation electrode (113) of the wearable sensor device (110) to deliver an initial stimulation to the nerve of the patient;
B. receiving the at least one physiological response and the nerve response from the at least one sensor device (111) and the at least one recording electrode (114) of the
wearable sensor device (110);
C. generating, based on the at least one physiological response and the nerve response, a response profile specific to the patient;
D. actuating the wearable sensor device (110) to deliver at least one subsequent stimulation by the at least one stimulation electrode (113) to the nerve of the patient according to the response profile;
E. receiving at least one subsequent physiological response and a subsequent nerve response from the at least one sensor device (111) and the at least one recording electrode (114) of the wearable sensor device (110);
F. adjusting, based on a change in the at least one subsequent physiological response and the subsequent nerve response, the response profile; and
G. repeating steps D-F until the at least one subsequent physiological response meets a target value indicative of management of the health condition.

2. The system (100) of claim 1, wherein the at least one physiological response comprises blood pressure, heart rate, heart rate variability, cardiac output, sustained hypertensive episodes, electrical brain activity, skeletal muscle function, electrical heart activity, respiration, oxygenation, chemical levels in blood, chemical levels in sweat, patient input, cerebral perfusion pressure optimization, or a combination thereof.

3. The system (100) of claim 1, wherein the computing device (120) is coupled to the sensor device (110) by a wired connection, a close-range wireless connection, a long-range wireless connection, or a combination thereof.

4. The system (100) of claim 1, wherein the computing device (120) comprises a personal computing device, a portable computing device, or a cloud computing device.

5. The system (100) of claim 1, wherein the memory component (124) further comprises a predictive artificial intelligence (AI) model configured to accept physiological responses and nerve responses as input and generate and adjust the response profile as output.

6. The system (100) of claim 1, wherein the nerve comprises a median nerve, a vagal nerve, or a sciatic nerve.

7. The system (100) of claim 1, wherein the health condition comprises recurrent pain, headache, hypertension, hypertensive events, heart attacks, strokes, anxiety, Post-Traumatic Stress Disorder (PTSD), treating clots/improve circulation, inflammation, motility, or a combination thereof.

8. A system (100) for real-time management of a health condition in a patient, the system (100) comprising:
a. a sensor device (110) coupled to a body of the patient, the device (110) comprising:
i. at least one sensor (111) configured to measure a blood pressure measurement of the patient and a heart rate measurement of the patient;
ii. a plurality of electrodes (112) comprising:
A. at least one stimulation electrode (113) configured to stimulate a nerve of the patient; and
B. at least one recording electrode (114) configured to record a nerve response from the nerve of the patient, generated in response to stimulation of the nerve; and
iii. a sensor processing module (115) communicatively coupled to the plurality of electrodes (112), configured to receive the blood pressure measurement, the heart rate measurement, and the nerve response from the at least one sensor (111) and the at least one recording electrode (114) and transmit the at least one physiological response and the nerve response to a computing device (120), and actuate the at least one stimulation electrode (113) to stimulate the median nerve; and
b. the computing device (120) communicatively coupled to the sensor device (110), comprising:
i. a processor (122) configured to execute computer-readable instructions; and
ii. a memory component (124) operatively coupled to the processor, comprising computer-readable instructions for:
A. actuating the at least one stimulation electrode (113) of the wearable sensor device (110) to deliver an initial stimulation to the median nerve of the patient;
B. receiving the blood pressure measurement, the heart rate measurement, and the nerve response from the at least one sensor device (111) and the at least one recording electrode (114) of the wearable sensor device (110);
C. generating, based on the blood pressure measurement, the heart rate measurement, and the nerve response, a response profile specific to the patient;
D. actuating the sensor device (110) to deliver at least one subsequent stimulation by the at least one stimulation electrode (113) to the median nerve of the patient;
E. receiving a subsequent blood pressure measurement, a subsequent heart rate measurement, and a subsequent nerve response from the at least one sensor device (111) and the at least one recording electrode (114) of the wearable sensor device (110);
F. adjusting, based on a change in the subsequent blood pressure measurement, the subsequent heart rate measurement, and the subsequent nerve response, the response profile; and
G. repeating steps D-F until the subsequent blood pressure measurement, the subsequent heart rate measurement, or a combination thereof meet a target value indicative of management of the health condition.

9. The system (100) of claim 8, wherein the computing device (120) is coupled to the sensor device (110) by a wired connection, a close-range wireless connection, a long-range wireless connection, or a combination thereof.

10. The system (100) of claim 8, wherein the computing device (120) comprises a personal computing device, a portable computing device, or a cloud computing device.

11. The system (100) of claim 8, wherein the memory component (124) further comprises a predictive artificial intelligence (AI) model configured to accept blood pressure measurements, heart rate measurements, and nerve responses as input and generate and adjust the response profile as output.

12. The system (100) of claim 8, wherein the health condition comprises recurrent pain, headache, hypertension, hypertensive events, heart attacks, strokes, anxiety, PTSD, treating clots/improve circulation, inflammation, motility, or a combination thereof.

13. A method for real-time management of a health condition in a patient, the method comprising:
a. providing a wearable sensor device (110) comprising:
i. at least one sensor (111);
ii. a plurality of electrodes (112); and
iii. a sensor processing module (115) communicatively coupled to the at least one sensor (111) and the plurality of electrodes (112);
b. applying the wearable sensor device (110) to a body of the patient such that the plurality of electrodes (112) are able to apply stimulation to and record responses from a nerve;
c. actuating, by a computing device (120), the wearable sensor device (110) to deliver an initial stimulation to the nerve;
d. measuring, by the at least one sensor (111), at least one physiological response;
e. measuring, by at least one electrode of the plurality of electrodes (112), a nerve response;
f. receiving, by the computing device (120), the at least one physiological response and the nerve response from the wearable sensor device (110);
g. generating, by the computing device (120), based on the at least one
physiological response and the nerve response, a response profile specific to the patient;
h. actuating, by the computing device (120), the wearable sensor device (110) to deliver at least one subsequent stimulation to the nerve of the patient according to the response profile;
i. measuring, by the at least one sensor (111), at least one subsequent physiological response;
j. measuring, by the at least one electrode of the plurality of electrodes (112), a subsequent nerve response;
k. receiving, by the computing device (120), the at least one subsequent physiological response and the subsequent nerve response from the wearable sensor device (110);
l. adjusting, by the computing device (120), based on a change in the at least one subsequent physiological response and the subsequent nerve response, the response profile; and
m. repeating steps h-l until the at least one subsequent physiological response meets a target value indicative of management of the health condition.

14. The method of claim 13, wherein the at least one physiological response comprises blood pressure, heart rate, heart rate variability, cardiac output, sustained hypertensive episodes, electrical brain activity, skeletal muscle function, electrical heart activity, respiration, oxygenation, chemical levels in blood, chemical levels in sweat, patient input, cerebral perfusion pressure optimization, or a combination thereof.

15. The method of claim 13, wherein the computing device (120) is coupled to the wearable sensor device (110) by a wired connection, a close-range wireless connection, a long-range wireless connection, or a combination thereof.

16. The method of claim 13, wherein the computing device (120) comprises a personal computing device, a portable computing device, or a cloud computing device.

17. The method of claim 13 further comprising:
a. providing a predictive artificial intelligence (AI) model configured to accept physiological responses and nerve responses as input and generate and adjust the response profile as output;
b. inputting the at least one physiological response and the nerve response into the predictive AI model;
c. generating, by the predictive AI model, the response profile;
d. inputting the at least one subsequent physiological response and the subsequent nerve response into the predictive AI model; and
e. adjusting, by the predictive AI model, the response profile.

18. The method of claim 13, wherein the nerve comprises a median nerve, a vagal nerve, or a sciatic nerve.

19. The method of claim 13, wherein the health condition comprises recurrent pain, headache, hypertension, hypertensive events, heart attacks, strokes, anxiety, PTSD, treating clots/improve circulation, inflammation, motility, or a combination thereof.
